(19) United States
(12) Patent Application Publication
(10) Pub. No.: US 2021/0213238 A1
(43) Pub. Date: Jul. 15, 2021

(54) MULTISENSORIAL AND MULTIMEDIA ROOM

(71) Applicant: HSIGN S.R.L., Monte Giberto (FM) (IT)

(72) Inventor: Maurizio ROSETTANI, Fermo (FM) (IT)

(73) Assignee: HSIGN S.R.L., Monte Giberto (FM) (IT)

(21) Appl. No.: 15/734,261

(22) PCT Filed: Jun. 15, 2018
(86) PCT No.: PCT/IB2018/054425

§ 371 Date: Dec. 2, 2020

(51) Int. Cl. A61M 21/02; A61M 21/00

(52) U.S. Cl. CPC A61M 21/02; A61M 21/0094; A61M 2021/005; A61M 2021/0027; A61M 2021/0016; A61M 2021/0066; A61M 2230/50; A61M 2205/3306; A61M 2230/06; A61M 2230/63; A61M 2230/10; A61M 2021/0072; A61M 2021/0055

(57) ABSTRACT

The present invention relates to a multi-sensory and multimedia room, such as a “smart room” or a multi-sensory room and in particular to a management system for managing such multi-sensory and multimedia room.

### FIELD OF THE INVENTION

The present invention relates to a multi-sensory and multimedia room or room and also to a management system for managing such multi-sensory and multimedia room.

### BACKGROUND

Environments that produce multi-sensory and multimedia stimuli on a user in order to obtain effects on the mind and body are widespread. Some examples of these environments are multi-sensory and/or multimedia rooms equipped with video, audio, sound and/or odour diffusion means and the like that are able, through the stimulations provided by the activation of such instruments, to produce effects on a user who spends time in and uses such rooms; for example, these means can be activated to try to bring the user into a relaxed state. Currently such environments are used at various levels, both for example for training in order to improve the efficacy of the learning systems through emotive suggestions that improve the receptive capacities, and for example in a medical/hospital setting to improve the state of some generative diseases, traumatic stress disorders, to have relaxing and therefore positive effects on patients with mental/behavioural disorders, etc. Finally, the use of these solutions in a working room is known, in order to increase wellbeing and promote concentration in the working room.

An example that falls within those mentioned that has a therapeutic function is the multi-sensory room such as Snoezelen, which is used in the field of therapy for people with cognitive disabilities, and is used as a non-verbal communication method, to improve relaxation and provide stimuli for people who would otherwise be difficult to “reach” emotionally and cognitively with other stimuli. Numerous studies have shown that different categories of patients, symptoms and disorders can take advantage from treatment with these stimulation solutions such as, for example:
people with physical disabilities;
people with cognitive disabilities;
people with psychiatric problems;
post-traumatic stress disorder;
autism;
acute and chronic pain control;
outcomes of strokes and traumatic brain injury.

A further example of application of these known technologies falls within the sector of suggestopedia, i.e. the methodology that allows the pupil to use their global receptive potential to learn concepts; it therefore falls within the field of multi-sensory learning.

It is however clear that such multimedia/multi-sensory systems can still be perfected from a technical point of view.

### SUMMARY OF THE INVENTION

The object of the invention is therefore that of fulfilling such objective by providing a multi-sensory and multimedia room with walls that contain technologies able to perform in a controlled and managed way all types of multi-sensory and multimedia stimulations, but also to provide the best reception conditions for the user, e.g. by bringing the user into a relaxed state, or a receptive and relaxed state. Under this condition, inherent to brain activities comprised between 4 and 10 Hz, the user's mind is readier and more predisposed to receive external information and/or indications.

These and other objects are reached by the multi-sensory and multimedia room according to the invention, the essential characteristics of which are defined by the first of the appended claims.

### BRIEF DESCRIPTION OF THE DRAWINGS

The characteristics and advantages of the multi-sensory and multimedia room according to the present invention will appear more clearly from the following description of an embodiment thereof, provided by way of non-limiting example with reference to the appended drawings wherein:

FIG. 1 is a schematic figure of the room according to the invention, representing a user inside it;

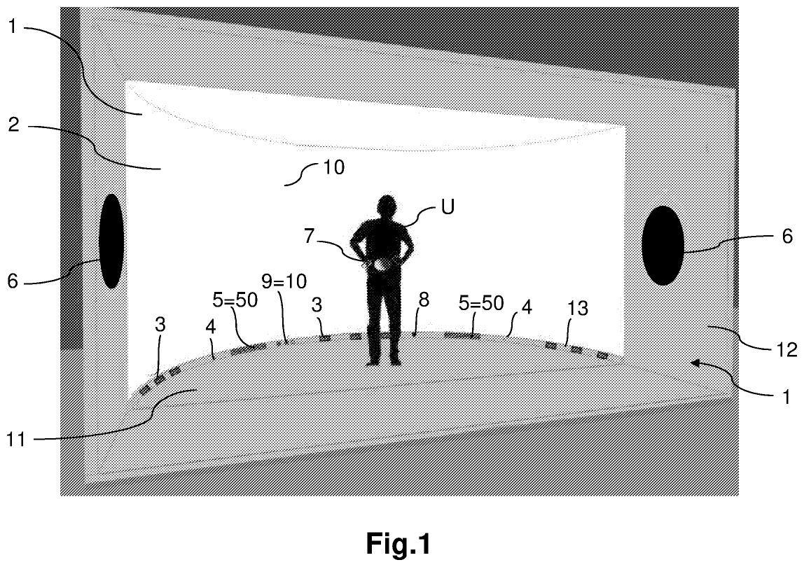

FIG. 2 is an operating diagram of the room according to the invention.

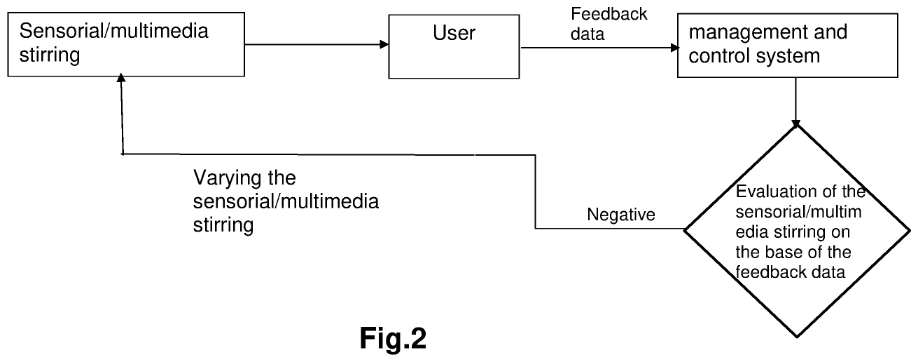

### DETAILED DESCRIPTION OF THE INVENTION

With reference to said figures, the multi-sensory and multimedia room 1 according to the invention adapted to house a user U is equipped at least with active means such as:
video means 2, adapted to transmit video images onto at least one wall delimiting such room;
audio means 3, adapted to emit sounds in such room 1;
olfactory means 4, adapted to emit odours and perfumed substances in such room;
climatic means 5, adapted to regulate the temperature of such room;
electromagnetic wave generating means 6, adapted at least to emit electromagnetic waves in such room suitable for performing frequency treatments;
   the room further comprising passive means such as:
sound reception means 8, adapted to receive sounds from such room;
movement and image acquiring means 9 adapted to acquire images from the room and movements of the user;
means for acquiring the user's vital signs 10;
   the room further comprising an electronic management and control system of such active means programmed to obtain feedback data continuously from such passive means, where the management and control system is programmed to autonomously evaluate the feedback data and, as a function of such autonomous evaluation, selectively activate and/or deactivate each of the active means and/or selectively vary the operation thereof continuously, the feedback data comprising one or more of the following:
verbal or sound signals emitted by the user;
movement signals or gestures or facial expressions performed by the user;
changes to the vital signs of the user;

In greater detail, the multi-sensory and multimedia room 1 according to the invention is delimited by a variable number of side walls. At least one of the side walls supports the aforementioned video means 2, such as, by way of non-limiting example, video screens composed of high resolution LED modules; in a preferred solution, the resolution, or the pixel, size of the individual LED is comprised between 0.9 and 1.2 mm².

The video means 2 can therefore affect the front side wall 10 (where front means the wall which, during use, is placed opposite the user), but also extend onto the adjacent side walls 12, i.e., still taking the user as a reference, the upper ceiling and the side walls. There is nothing to prevent the video means also extending to the floor 11 and to the wall behind the user.

The projection can take place on a flat but also a curved surface for a greater sensation of immersion into the image.

The video means 2 are able to represent stationary but also moving images, texts, drawings, colours and coloured lights to provide for colour therapy treatments.

The room is also equipped with audio means 3 comprising, for example, a plurality of acoustic diffusers. The diffusers are arranged in the room to as to give the user the sensation of a sound immersion experience, i.e., they are arranged possibly at 360° around the user. Again, the audio means are further able to emit sound waves that also contain frequencies outside the audible field, in particular infrasound from 1 Hz to 10 Hz.

With regard to the olfactory means 4, they comprise an essence diffusion system. This is completed with a tank of essences to be vaporized into the room which is provided at the edge of the room itself and with a plurality of vaporizer nozzles, distributed in the room and adapted to be connected to the tank for taking the essences to be vaporized into the room and diffusing them. Advantageously, such diffusion system and in general such plurality of nozzles can be operatively associated with the climatic means 5. In more detail, such operating association consists of activating or varying the operation of the climatic means 5 to promote the diffusion of the essence or on the contrary to remove its odour in the room when treatment with the essence is to be interrupted.

The climatic means 5 comprise a climate control system equipped with an ionizer with openings 50 sized according to the volume of the room. The system can handle the intensity of the air flow introduced (e.g., by stimulating a breeze or a wind) or suck the air with a suitable flow rate to remove the olfactory content in a short time. It can also simulate atmospheric events such as fog. As just mentioned, the system is equipped with an ionizer to modify the conditions of the air introduced according to the images transmitted in the video (e.g., if the images relate to a mountain landscape then the ionizer will be activated to introduce “cleaner” air into the room through the introduction of negative ions).

Again, the room according to the invention is equipped with electromagnetic wave generating means 6 that through Tesla coil antennae, represented schematically in the figure, are transferred into the room.

Possibly, in some specific applications, further electromagnetic wave generating means of the portable type 7 may be provided, which are adapted to generate and transmit electromagnetic waves from 0.1 Hz to 10 MHz, through relevant cables and bracelets connected to the user and equipped with connectors to allow the passage of microcurrents directly in contact with the body.

It is known that the propagation of electromagnetic waves into the room in which a user is immersed at certain frequencies can influence their brain response, obtaining a series of benefits not only on the state of relaxation but also on other symptoms such as pain, stress, etc. In the solution shown in the figures, there are two Tesla type coil antennae. Obviously, this does not exclude the possibility of there being a larger number of them.

In an embodiment as illustrated in the figures, at the base of the front side wall and in general at the base of the vertical walls, between the floor and them, a baseboard 13 runs where the aforementioned active means are inserted, and in particular the audio means, the olfactory means and the climatic means. Possibly and preferably in the baseboard, part of the passive means is also inserted, such as at least the image acquisition means and the sound reception means.

Those described above are the so-called active means of the room, as they exert actions directly on the user or however in the room and secondly on the user.

The multi-sensory and multimedia room according to the invention further comprises passive means, i.e. means that are not directly active on the user but have the function of receiving information/signals from him/her.

The passive means comprise sound reception means 8, adapted to receive sounds from the room and in particular from the user, movement and image acquisition means 9 adapted to acquire images from the room and movements of the user, acquisition means of the user's vital signs 10.

In more detail, the passive means listed above have the purpose of continuously monitoring the user's activity and their perception of the stimuli sent by the active means.

Again, the sensory room comprises an electronic control system operatively connected to the passive means and to the active means, such as to continuously receive feedback on the state of the user from the passive means and autonomously vary the operation or selectively activate/deactivate the active means based on such feedback. Such feedback can be identified in one or more of the following categories:
verbal or sound signals emitted by the user;
movement signals or gestures or facial expressions performed by the user;
changes in the user's vital signs;
changes in the user's brain activity.

In particular, the verbal or sound signals emitted by the user are received by the aforementioned sound reception means 8 that comprise one or more environmental microphones, preferably inserted in the aforementioned baseboard. The movement signals or gestures or facial expressions performed by the user are acquired through the image acquisition means 9, comprising at least one television camera. The image acquisition means 9 may further comprise one or more movement sensors.

Movements or gestures obviously also mean changes in the user's posture. Possibly the image and movement acquisition means also comprise an infra-red television camera.

The vital parameter acquisition means 10 comprise sensors (also positioned on the actual user) but can also make use of the aforementioned at least one television camera and at least one movement sensor; they detect at least the user's heart rate and body temperature.

In a further embodiment, the passive means also comprise means for monitoring the user's brain activity adapted to monitor the electromagnetic waves emitted by the brain activity in progress. For example, such means comprise sensors for detecting the user's electroencephalic or cerebral waves.

A further aspect of the invention relates to a method for managing the multi-sensory and multimedia room described above. In particular, the method comprises the steps of:
providing at least one multi-sensory/multimedia stimulus to a user through one or more of the active means;
continuously detecting feedback data on the user through the passive means in relation to at least one stimulus;
sending the feedback data continuously to the management and control system;
evaluating the positivity of the user's reaction to such at least one stimulus;

if such evaluation is negative, modifying through the management and control system the at least one stimulus by intervening on the functionality of the active means.

Therefore, the system responds continuously and autonomously to the user's needs. That is, it does not need a direct command for interpreting their needs but evaluates their reactions and based on them the functionality of the active means for having positive emotional feedback. Modifying the functionality of the active means indicates activating and/or deactivating them but also modifying the essences emitted, or the sounds or videos replicated in the multimedia and multi-sensory room, like modifying the temperature of the room or the humidity percentage.

Again, the evaluation of the positive response to the stimulus is obtained from the system through the evaluation of the data listed above, i.e., gestures, movements, facial expressions, vital signs, sounds emitted by the user.

For example, expressions of disgust or fear can be recognized by the system and interpreted as negative. An increase in body temperature beyond a certain desired threshold is a signal of a negative response to the sensory stimulus.

One or more of the following events, provided by way of non-limiting example, are indicative of a negative response to a sensory stimulus:
shouting;
rash gestures;
facial expressions connected with fear, disgust;
verbalizations of words that can be interpreted as signals of interruption (e.g.: “enough”, “stop”);
body temperature greater than 38° or less than 34°
heart rate over 120 beats per minute.

The multi-sensory and multimedia room according to the invention obtains a series of new advantages with respect to known solutions. In particular, thanks to the continuous feedback provided by the user, the control system is able to modify, in real time and continuously, the stimulations provided to the actual user, to obtain through them the desired conditioning result.

The room according to the invention is therefore adapted to be used also for therapeutic or wellbeing purposes since by applying certain protocols it will be possible to realize specific stimulations for each user for the purpose of modifying, for example, the vital signs and brain activity from the perspective of bringing him/her to a condition of relaxation and/or cognitive receptivity.

The active interaction of the system with the user is a new functionality of the room according to the invention with respect to known multi-sensory/multimedia instruments. In particular, the room according to the invention combines means for preparing the user in suitable mental conditions before performing the stimuli, which are subject to verification through the feedback signals listed and described above. The active means are therefore controlled by modifying their stimulation functionalities in real time and following the analysis of the data on the emotional reactions of the subject in response to the stimuli or for other contingent requirements of the actual user.

Other variants and/or modifications may be made to the multimedia and multi-sensory room according to the invention, but without thereby departing from the scope of protection of the invention itself as defined by the appended claims.

1. A multi-sensory and multimedia room adapted to house a user,
delimited by perimeter walls equipped with active means selected from the group consisting of:
video means, adapted to transmit video images onto at least one of said perimeter walls;
audio means, adapted to emit sounds in said room;
olfactory means, adapted to emit odours and perfumed substances in said room;
climatic means, adapted to regulate the temperature of said room;
electromagnetic wave emitting means, adapted at least to emit electromagnetic waves in said room;
said room further comprising passive means such as:
sound reception means, adapted to receive sounds from said room;
movement and image acquisition means adapted to acquire images from said room and movements of said user;
means for acquiring said user's vital signs;
said room further comprising an electronic management and control system of said active means programmed to obtain feedback data continuously from said passive means, where said management and control system is programmed to autonomously evaluate said feedback data and, as a function of said autonomous evaluation, selectively activate and/or deactivate each of said active means and/or selectively vary the operation thereof continuously, said feedback data comprising one or more of the following:
verbal or sound signals emitted by the user;
movement signals or gestures or facial expressions performed by the user;
changes in the user's vital signs.

2. The room according to claim 1 wherein said passive means further comprise means for monitoring the user's brain activity.

3. The room according to claim 2 wherein said feedback data further comprise changes in the user's brain activity.

4. The room according to claim 1 wherein said active means further comprise further means for generating electromagnetic waves adapted to perform frequency treatments and to be worn by the user.

5. The room according to claim 4 wherein said further electromagnetic wave generator means comprise cables or bracelets connected to the user such as to allow the passage of microcurrents directly onto the user's body, such as electromagnetic waves from 0.1 Hz to 10 MHz.

6. The room according to claim 1, wherein said video means comprise video screens composed of LED modules with a resolution comprised between 0.9 and 1.2 mm².

7. The room according to claim 1, wherein said video means can affect one or more of said perimeter walls.

8. The room according to claim 1, wherein said audio means are adapted to emit sound waves that contain frequencies outside the audible field.

9. The room according to claim 8 wherein said sound frequencies are comprised between 1 Hz and 10 Hz.

10. The room according to claim 8 wherein said audio means comprise a plurality of diffusers scattered in said room.

11. The room according to claim 1 wherein said olfactory means comprise an essence diffusion system and a plurality of vaporizer nozzles.

12. The room according to claim 1 wherein said climatic means comprise a climate control system with an ionizer.

13. The room according to claim 12 wherein said climate control system is operatively connected to said olfactory means.

14. The room according to claim 1 wherein said active means can be inserted in a baseboard (13) realized between said perimeter walls and a floor of said room.

15. The room according to claim 1 wherein said wearable means comprise a bracelet provided with electrodes connected with a wearable device adapted to perform frequency treatments, adapted to generate microcurrents modulated with frequencies comprised between 0.1 Hz and 10 MHz.

16. The room according to claim 1 wherein said sound reception means comprise at least one environmental microphone.

17. The room according to claim 1 wherein said image acquisition means comprise at least one television camera.

18. The room according to claim 17 wherein said at least one television camera is an infra-red television camera.

19. The room according to claim 17 wherein said image acquisition means further comprise one or more movement sensors.

20. The room according to claim 1 wherein said vital sign acquisition means are adapted to detect at least the body temperature and heart rate of the user.

21. Method for managing a multi-sensory and multimedia room according to claim 1, comprising the following steps:
providing at least one multi-sensory/multimedia stimulus to a user through one or more of said active means;
continuously detecting feedback data on the user through said passive means in relation to said at least one stimulus;
sending said feedback data continuously to said management and control system;
said management and control system autonomously evaluating the positivity of the reaction of said user to said at least one stimulus;
if such evaluation is negative said management and control system autonomously modifies said at least one stimulus by intervening on the functionality of said active means.

22. The method according to claim 21 where said evaluation is considered negative if said feedback data comprise one or more of the following reactions of the user:
shouting;
rash gestures;
facial expressions connected with fear, disgust;
verbalizations of words that can be interpreted as signals of interruption;
body temperature greater than 38° or less than 34°
heart rate over 120 beats per minute.
